SEQUENCE LISTING (27 sequences)

SEQ ID NO: 1 - DNA, 861 bp, Homo sapiens
  CDS (58)..(717)
  5′UTR (1)..(57)
  3′UTR (718)..(861)
  sig_peptide (58)..(174)
  mat_peptide (298)..(717)
  mat_peptide (370)..(717)
  mat_peptide (379)..(717)
  misc_structure (661)..(663): CARBOHYD glycosylated asparagine at Asn122
aggagggtgggggaacagctcaacaatggctgatgggcgctcctggtgttgatagagatggaacttggacttggaggcctctccacgctgtcccactgcccctggcctaggcggcagcctgccctgtggcccaccctggccgctctggctctgctgagcagcgtcgcagaggcctccctgggctccgcgccccgcagccctgccccccgcgaaggccccccgcctgtcctggcgtcccccgccggccacctgccggggggacgcacggcccgctggtgcagtggaagagcccggcggccgccgccgcagccttctcggcccgcgcccccgccgcctgcacccccatctgctcttccccgcgggggccgcgcggcgcgggctgggggcccgggcagccgcgctcgggcagcgggggcgcggggctgccgcctgcgctcgcagctggtgccggtgcgcgcgctcggcctgggccaccgctccgacgagctggtgcgtttccgcttctgcagcggctcctgccgccgcgcgcgctctccacacgacctcagcctggccagcctactgggcgccggggccctgcgaccgcccccgggctcccggcccgtcagccagccctgctgccgacccacgcgctacgaagcggtctccttcatggacgtcaacagcacctggagaaccgtggaccgcctctccgccaccgcctgcggctgcctgggctgagggctcgctccagggctttgcagactggacccttaccggtggctcttcctgcctgggaccctcccgcagagtcccactagccagcggcctcagccagggacgaaggcctcaaagctgagaggcccctaccggtgggtgatg

SEQ ID NO: 2 - protein, 220 aa, Homo sapiens
MELGLGGLSTLSHCPWPRRQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 3 - DNA, 2136 bp, Murinae gen. sp.
  CDS (975)..(1646)
  5′UTR (1)..(974)
  3′UTR (1647)..(2136)
  sig_peptide (975)..(1091)
  mat_peptide (1215)..(1646)
  mat_peptide (1290)..(1646)
  mat_peptide (1299)..(1646)
gcggccgcgaattcggcacgagggcgtctcgctgcagcccgcgatctctactctgcctcctggggtcttctccaaatgtctagcccccacctagagggacctagcctagccagcggggaccggatccggagggtggagcggccaggtgagccctgaaaggtggggcggggcgggggcgctctgggccccaccccgggatctggtgacgccggggctggaatttgacaccggacggcggcgggcaggaggctgctgagggatggagttgggctcggcccccagatgcggcccgcgggctctgccagcaacaagtccctcgggccccagccctcgctgcgactggggcttggagccctgcacccaagggcacagaccggctgccaaggccccacttttaactaaaagaggcgctgccaggtgcacaactctgggcatgatccacttgagcttcgggggaaagcccagcactggtcccaggagaggcgcctagaaggacacggaccaggacccctttggtatggagtgaacgctgagcatggagtggaaggaactcaagttactactttctccaaccaccctggtaccttcagccctgaagtacagagcagaagggtcttagaagacaggaccacagctgtgtgagtctcccccctgaggccttagacgatctctgagctcagctgagctttgtttgcccatctggagaagtgagccattgattgaccttgtggcatcgcgaaggaacaggtcctgccaagcacctaacacagagagcaaggttctccatcgcagctaccgctgctgagttgactctagctactccaacctcctgggtcgcttcgagagactggagtggaaggaggaataccccaaaggataactaactcatctttcagtttgcaagctgccgcaggaagagggtggggaaacgggtccacgaaggcttctgatgggagcttctggagccgaaagctatggaactgggacttgcagagcctactgcattgtcccactgcctccggcctaggtggcagtcagcctggtggccaaccctagctgttctagccctgctgagctgcgtcacagaagcttccctggacccaatgtcccgcagccccgccgctcgcgacggtccctcaccggtcttggcgccccccacggaccacctgcctgggggacacactgcgcatttgtgcagcgaaagaaccctgcgacccccgcctcagtctcctcagcccgcacccccgccgcctggtcccgcgctccagtctcctcccgctgcgctccgcggggcacgcgcggcgcgtgcaggaacccggagcagccgcgcacggaccacagatgcgcgcggctgccgcctgcgctcgcagctggtgccggtgagcgcgctcggcctaggccacagctccgacgagctgatacgtttccgcttctgcagcggctcgtgccgccgagcacgctcccagcacgatctcagtctggccagcctactgggcgctggggccctacggtcgcctcccgggtcccggccgatcagccagccctgctgccggcccactcgctatgaggccgtctccttcatggacgtgaacagcacctggaggaccgtggaccacctctccgccactgcctgcggctgtctgggctgaggatgatctatctccaagcctttgcacactagacccatgtgttgccctacctggaacagctccaccgggcctcactaaccaggagcctcaactcagcaggatatggaggctgcagagctcaggccccaggccggtgagtgacagacgtcgtcggcatgacagacagagtgaaagatgtcggaaccactgaccaacagtcccaagttgttcatggatcccagctctacagacaggagaaacctcagctaaagagaactcctctgggagaatccagaaatggccctctgtcctggggaatgaattttgaagagatatatatacatatatacattgtagtcgcgttgctggaccagcctgtgctgaaaccagtcccgtgttcacttgtggaagccgaagccctatttattatttctaaattatttatttactttgaaaaaaaacggccaagtcggcctccctttagtgagggttaatttgtgatcccggg

SEQ ID NO: 4 - protein, 224 aa, Murinae gen. sp.
MELGLAEPTALSHCLRPRWQSAWWPTLAVLALLSCVTEASLDPMSRSPAARDGPSPVLAPPTDHLPGGHTAHLCSERTLRPPPQSPQPAPPPPGPALQSPPAALRGARAARAGTRSSRARTTDARGCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG

SEQ ID NO: 5 - protein, 224 aa, Rattus sp.
  SIGNAL (1)..(39)
  PROPEP (40)..(80)
  PEPTIDE (81)..(224)
  PEPTIDE (109)..(224)
  PEPTIDE (112)..(224)
  CARBOHYD (206)
  DISULFID (127)..(192)
  DISULFID (154)..(220)
  DISULFID (158)..(222)
  DISULFID (191): Interchain disulfide link
MELGLGEPTALSHCLRPRWQPALWPTLAALALLSSVTEASLDPMSRSPASRDVPSPVLAPPTDYLPGGHTAHLCSERALRPPPQSPQPAPPPPGPALQSPPAALRGARAARAGTRSSRARATDARGCRLRSQLVPVSALGLGHSSDELIRFRFCSGSCRRARSPHDLSLASLLGAGALRSPPGSRPISQPCCRPTRYEAVSFMDVNSTWRTVDHLSATACGCLG

SEQ ID NO: 6 - protein, 197 aa, Homo sapiens
MQRWKAAALASVLCSSVLSIWMCREGLLLSHRLGPALVPLHRLPRTLDARIARLAQYRALLQGAPDAMELRELTPWAGRPPGPRRRAGPRRRRARARLGARPCGLRELEVRVSELGLGYASDETVLFRYCAGACEAAARVYDLGLRRLRQRRRLRRERVRAQPCCRPTAYEDEVSFLDAHSRYHTVHELSARECACV

SEQ ID NO: 7 - protein, 156 aa, Homo sapiens
MAVGKFLLGSLLLLSLQLGQGWGPDARGVPVADGEFSSEQVAKAGGTWLGTHRPLARLRRALSGPCQLWSLTLSVAELGLGYASEEKVIFRYCAGSCPRGARTQHGLALARLQGQGRAHGGPCCRPTRYTDVAFLDDRHRWQRLPQLSAAACGCGG

SEQ ID NO: 8 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 9 - DNA, 39 bp, Homo sapiens
aaggaaaaaagcggccgccatggaacttggacttggagg

SEQ ID NO: 10 - DNA, 39 bp, Homo sapiens
ttttttccttggcggccgctcagcccaggcagccgcagg

SEQ ID NO: 11 - protein, 140 aa, Homo sapiens
  CARBOHYD (122): glycosylated asparagine
PPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 12 - protein, 116 aa, Homo sapiens
  CARBOHYD (98): glycosylated asparagine
AARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 13 - protein, 113 aa, Homo sapiens
  CARBOHYD (95): glycosylated asparagine
AGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 14 - protein, 112 aa, Homo sapiens
GGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 15 - protein, 111 aa, Homo sapiens
GPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 16 - protein, 110 aa, Homo sapiens
PGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 17 - protein, 109 aa, Homo sapiens
GSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 18 - protein, 108 aa, Homo sapiens
SRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 19 - protein, 107 aa, Homo sapiens
RARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 20 - protein, 106 aa, Homo sapiens
ARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 21 - protein, 105 aa, Homo sapiens
RAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 22 - protein, 104 aa, Homo sapiens
AAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 23 - protein, 103 aa, Homo sapiens
AGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 24 - protein, 102 aa, Homo sapiens
GARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 25 - protein, 101 aa, Homo sapiens
ARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 26 - protein, 100 aa, Homo sapiens
RGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 27 - protein, 99 aa, Homo sapiens
GCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

(19) United States
(12) Patent Application Publication
(10) Pub. No.: US 2012/0083453 A1
(43) Pub. Date: Apr. 5, 2012

(54) TREATMENT USING NEUBLASTIN POLYPEPTIDES

(75) Inventor: Dinah W.Y. Sah, Boston, MA (US)

(73) Assignee: BIOGEN IDEC MA INC., Cambridge, MA (US)

(21) Appl. No.: 13/012,408

(22) Filed: Jan. 24, 2011

Related U.S. Application Data
(63) Continuation of application No. 12/399,777, filed on Mar. 6, 2009, now abandoned.
Application No. 12/399,777 is a continuation of application No. 10/669,853, filed on Sep. 24, 2003, now abandoned.
Application No. 10/669,853 is a continuation of application No. PCT/US02/06388, filed on Feb. 28, 2002.
(60) Provisional application No. 60/287,554, filed on Mar. 28, 2001.

(51) Int. Cl. A61K 38/17; A61P 25/00

(52) U.S. Cl. 514/18.3

(57) ABSTRACT

The invention relates to treatments of neuropathic pain, including tactile allodynia, and to treatments for reducing loss of pain sensitivity associated with neuropathy. The present treatments involve the use of neublastin (NBN) polypeptides.

This application is a continuation of International Patent Application No. PCT/US02/06388 filed Feb. 28, 2002, and claims priority to U.S. Provisional Application Ser. No. 60/287,554, filed Mar. 28, 2001.

### FIELD OF THE INVENTION

The invention relates to treatments for neuropathic pain, including tactile allodynia, and to treatments for reducing loss of pain sensitivity associated with neuropathy.

### BACKGROUND OF THE INVENTION

Neuropathic pain is a category of pain that includes several forms of chronic pain and which results from dysfunction of nervous rather than somatic tissue. Neuropathic pain, that is pain deriving from dysfunction of the central or peripheral nervous system, may also be a consequence of damage to peripheral nerves or to regions of the central nervous system, may result from disease, or may be idiopathic. Symptoms of neuropathic pain include sensations of burning, tingling, electricity, pins and needles, stiffness, numbness in the extremities, feelings of bodily distortion, allodynia (pain evoked by stimulation of the skin that is normally innocuous), hyperalgesia (abnormal sensitivity to pain), and hyperpathia (an exaggerated pain response persisting long after the pain stimuli cease).

Several common causes of neuropathic pain are diabetes, cancer chemotherapy, herpes zoster infection, cervical or lumbar root compression owing to degenerative spine disease, malignant lesions of nerve plexus or root, nerve degeneration, such as from amputation, HIV infection, and lesions of central pain pathways, including the spinothalamic tract, thalamus, or thalamic radiations. Additional causes of neuropathic pain include drug-induced, or toxin-induced neuropathies. For example, antivirals such as ddI, ddC and d4T commonly cause peripheral neuropathies, as do phenyloin (a seizure medication), isoniazid (a tuberculosis medication), vincristine, vinblastine, taxol, taxotere and cisplatin (cancer chemotherapeutic agents), high dose vitamins, and folic acid antagonists.

Current therapies for the management of neuropathic pain are of limited benefit to many patients, and involve undesirable side effects or dose-limiting toxicities. In addition, current therapies are symptomatic, not disease modifying. Needs remain for improved therapies for the management and treatment of neuropathic pain, especially those that target the underlying pathology.

### SUMMARY OF THE INVENTION

This invention provides improved methods for treating neuropathic pain, for treating tactile allodynia and for reducing loss of pain sensitivity associated with neuropathy. The present methods use neublastin (“NBN”) polypeptides, including full-length neublastin polypeptides or bioactive truncated neublastin polypeptides, including, e.g., at least SEQ ID NOS:2, 4, 5 and 11-27. In addition, the invention provides pharmaceutical compositions containing a full-length neublastin polypeptide or a truncated neublastin polypeptide suspended, dissolved, or dispersed in a pharmaceutically acceptable carrier.

In a specific embodiment, the neublastin polypeptide may be any polypeptide of AA₈₀-AA₁₄₀ of SEQ ID NO:2, AA₄₁-AA₁₄₀ of SEQ ID NO:2, AA₁-AA₁₄₀ of SEQ ID NO:2, AA₂₅-AA₁₄₀ of SEQ ID NO:2, AA₂₈-AA₁₄₀ of SEQ ID NO:2, AA₋₈₀-AA₁₄₄ of SEQ ID NO:4, AA₁-AA₁₄₄ of SEQ ID NO:4, AA₁-AA₂₂₄ of SEQ ID NO:5, or AA₈₁-AA₂₂₄ of SEQ ID NO:5; at least one polypeptide comprising the C-terminal sequence set forth in either AA₁₀₇-AA₁₄₀ of SEQ ID NO:2 or AA₇₆-AA₁₄₀ of SEQ ID NO:2, and which retain the seven Cys residues characteristic of the GDNF family and of the TGF-beta super family; at least one polypeptide comprising SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26 or SEQ ID NO:27; or at least one polypeptide sequence that has greater than 70% amino acid homology to a sequence listed above.

The neublastin polypeptide may be modified by a derivative moiety to have an extended residence time and or increased concentration in the body. The neublastin polypeptides may be N-glycosylated neublastin polypeptides. In addition, the neublastin polypeptide may be derivatized with one or more moieties including, but not limited to, polyethylene glycol moieties, aliphatic esters, amides, N-acyl-derivatives, or O-acyl derivatives.

In one embodiment, the invention features a method for treating neuropathic pain in a subject comprising administering to the subject an effective amount of a neublastin polypeptide, including, e.g., any one of SEQ ID NOS:2, 4, 5 and 11-27, either alone, or by also administering to the subject an effective amount of an analgesia-inducing compound selected from the group consisting of opioids, anti-arrhythmics, topical analgesics, local anaesthetics, anticonvulsants, antidepressants, corticosteroids and non-steroidal anti-inflammatory drugs (NSAIDS). In a preferred embodiment, the analgesia-inducing compound is an anticonvulsant. In another preferred embodiment, the analgesia-inducing compound is gabapentin ((1-aminomethyl)cyclohexane acetic acid) or pregabalin (S-(+)-4-amino-3-(2-methylpropyl) butanoic acid).

In another embodiment, the invention features a method for treating tactile allodynia in a subject, either by administering to the subject an effective amount of a neublastin polypeptide, including, e.g., at least one of SEQ ID NOS:2, 4, 5 and 11-27, either alone, or by administering to the subject an effective amount of a neublastin polypeptide with an effective amount of an analgesia-inducing compound selected from the group consisting of opioids, anti-arrhythmics, topical analgesics, local anaesthetics, anticonvulsants, antidepressants, corticosteroids and NSAIDS. In a preferred embodiment, the analgesia-inducing compound is an anticonvulsant. In another preferred embodiment, the analgesia-inducing compound is gabapentin ((1-aminomethyl)cyclohexane acetic acid) or pregabalin (S-(+)-4-amino-3-(2-methylpropyl)butanoic acid).

Neublastin polypeptide may be administered in association with a therapeutic agent, including but not limited to an anti-cancer agent or an anti-viral agent. Anti-cancer agents include, but are not limited to, taxol, taxotere, cisplatin, nocodazole, vincristine, vindesine and vinblastine. Anti-viral agents include, but are not limited to, ddI, DDC, d4T, foscarnet, dapsone, metronidazole, and isoniazid.

The invention includes a method for treating neuropathic pain in a subject. In a specific embodiment, the neuropathic pain is associated with diabetic neuropathy. In another embodiment, the neuropathic pain is associated with infection of a subject by a virus, including but not limited to a herpes virus, a human immunodeficiency virus (HIV), and a papilloma virus. Neuropathic pain may be associated with infection by a herpes zoster virus, or especially with post-herpetic neuralgia. In a further embodiment, the neuropathic pain is associated with sciatica. In another embodiment, the invention features a method for modulating the loss of pain sensitivity in a subject afflicted with a neuropathy. In a preferred embodiment, the neuropathy is diabetic neuropathy. In another preferred embodiment, the loss of pain sensitivity is a loss in thermal pain sensitivity.

In further embodiments, the neuropathic pain is hyperalgesic pain, phantom pain, thermal hyperalgesia, or due to injury associated with trauma. In addition, neuropathic pain may also be associated with hereditary neuropathy (including but not limited to Friedreich ataxia, familial amyloid polyneuropathy, Tangier disease, Fabry disease), metabolic disorders (including but not limited to renal insufficiency and hypothyroidism), vitamin deficiencies (including but not limited to vitamin B12 deficiency, vitamin B6 deficiency, and vitamin E deficiency), toxic and iatrogenic neuropathies (including but not limited to alcoholism, vitamin B6 intoxication, hexacarbon intoxication, amiodarone, chloramphenicol, disulfuram, isoniazide, gold, lithium, metronidazole, misonidazole, nitrofurantoin), infectious neuropathies (including but not limited to leprosy, Lyme disease), auto-immune neuropathies (including but not limited to Guillain-Barre syndrome, chronic inflammatory de-myelinating polyneuropathy, monoclonal gammopathy of undetermined significance and polyneuropathy), trigeminal neuralgia, entrapment syndromes (including but not limited to Carpel tunnel), post-traumatic neuralgia, phantom limb pain, multiple sclerosis pain, complex regional pain syndromes (including but not limited to reflex sympathetic dystrophy, causalgia), neoplasia, vasculitic/angiopathic neuropathy and idiopathic neuropathy.

The foregoing methods contemplate administering to the subject, preferably systemically, a formulation comprising a neublastin polypeptide at a dosage of between 1 μg/kg to 30,000 μg/kg body weight of the subject, per dose. In alternative embodiments, the dosage is between 10 μg/kg to 30,000 μg/kg body weight of the subject, per dose; between 10 μg/kg to 10,000 μg/kg body weight of the subject, per dose; between 25 μg/kg to 10,000 μg/kg body weight of the subject, per dose; between 25 μg/kg to 3,000 μg/kg body weight of the subject, per dose; and between 50 μg/kg to 3,000 μg/kg body weight of the subject, per dose.

The neublastin polypeptide used in the foregoing methods can be administered via any suitable delivery system, and preferably from the group consisting of intravenous delivery, intramuscular delivery, intrapulmonary delivery, subcutaneous delivery, and intraperitoneal delivery, most preferably via intramuscular delivery or subcutaneous delivery.

The neublastin polypeptide used in the foregoing methods can also be administered via intrathecal delivery.

The NBN polypeptide-containing formulation of the invention may be administered in a timed-released composition.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a broken line plot illustrating near complete prevention of tactile allodynia by subcutaneous neublastin (NBN) in rats with L5/L6 spinal nerve ligation (SNL);

FIG. 2 is a broken line plot illustrating near complete prevention of thermal hyperalgesia by subcutaneous neublastin (NBN) in rats with L5/L6 spinal nerve ligation (SNL).

FIG. 3 is a broken line plot illustrating the near complete reversal of fully established tactile allodynia by subcutaneous neublastin (NBN) in rats with L5/L6 spinal nerve ligation (SNL);

FIG. 4 is a broken line plot illustrating the near complete reversal of fully established thermal hyperalgesia by subcutaneous neublastin (NBN) in rats with L5/L6 spinal nerve ligation (SNL).

FIG. 5 is a bar graph illustrating near complete normalization of thermal hypoalgesia by subcutaneous neublastin in rats with STZ (streptozotocin)-induced neuropathy.

FIG. 6A and FIG. 6B are graphic representations illustrating the prevention of thermal hyperalgesia (FIG. 6A), prevention of thermal hypoalgesia (FIG. 6B), and reversal of thermal hyperalgesia (FIG. 6B) by subcutaneous neublastin in rats with STZ (streptozotocin)-induced neuropathy at 4 weeks (FIG. 6A) and 8 weeks (FIG. 6B) post STZ treatment.

FIG. 7 is a broken line plot illustrating dose-dependent neublastin (NBN) reversal of fully established tactile allodynia by subcutaneous neublastin in rats with L5/L6 spinal nerve ligation (SNL).

FIG. 8 is a broken line plot illustrating dose-dependent neublastin (NBN) reversal of fully established thermal hyperalgesia by subcutaneous neublastin in rats with L5/L6 spinal nerve ligation (SNL).

### DETAILED DISCLOSURE OF THE INVENTION

This invention relates to methods and compositions for treating neuropathic pain, for treating tactile allodynia and for reducing loss of pain sensitivity by administering a neublastin polypeptide to a subject at risk of, or afflicted with, neuropathic pain.

Neublastin polypeptides are proteins which promote survival, maintain phenotypic differentiation, prevent degeneration, promote regeneration, and restore the activity of neuronal cells and tissues. Neublastin (initially described, e.g., in WO 00/01815) has alternately been referred to as “artemin” (see, e.g., WO 00/18799) and “enovin” (see, e.g., WO 00/04050). Neublastin has been classified as a distant member of the TGF-β superfamily (Massague, et al., 1994, Trends in Cell Biology, 4:172-178) and is a member of glial cell line-derived neurotrophic factor ligand family (“GDNF”; WO 93/06116, incorporated herein by reference), in the family which includes GDNF, persephin (“PSP”; Milbrandt et al., 1998, Neuron 20:245-253, incorporated herein by reference) and neurturin (“NTN”; WO 97/08196, incorporated herein by reference). The ligands of the GDNF subfamily have in common their ability to induce signalling through the RET receptor tyrosine kinase. These three ligands of the GDNF subfamily differ in their relative affinities for a family of neurotrophic receptors, the GFRα receptors. Neublastin acts preferably through the GFRα3—RET complex. Baudet et al., Development, 127:4335-44 (2000); Baloh et al., Neuron, 21:1291-1302 (1998); Airaksinen et al., Mol. Cell. Neuroscience, 13:313-325 (1999).

An amino acid sequence comparison of Neublastin (SEQ ID NO:2) to the GDNF subfamily members Neurturin (SEQ ID NO:6), Persephin (SEQ ID NO:7) and GDNF (SEQ ID NO:8) is shown in Table 1. Neublastin polypeptides useful in this invention preferably hold the GDNF subfamily fingerprint, i.e. the amino acid residues underlined in Table 1.

TABLE 1
Amino Acid Sequence Comparison of Neublastin (SEQ ID NO: 2)
to Neurturin (SEQ ID NO: 6), Persephin (SEQ ID NO: 7),
and GDNF (SEQ ID NO: 8)

| | |
|---|---|
| Neurturin-full | --------------------MQRWKAAALASVLCSSVLSIWMCREGLLLSHRLGPA |
| Neublastin | MELGLGGLSTLSHCPWPRRQPALWPTLAALALLSSVAEASLGSAPRSPAPREGPPP |
| Persephin-full | -------------------------------------------------------- |
| GDNF_HUMAN-full | -----MKLWDVVAVCLVLLHTASAFPLPAGKRPPEAPAEDRSLGRRRAPFALSSDS |
| Neurturin-full | LVPLHRLPRTLDARIARLAQYRALLQGAPDAMELRELTPWAGRPPGPRRRAGPRRR |
| Neublastin | VLASPAGHLPGGRTARWCSGRARRPPPQPSRPAPPPPAPPSALPRGGRAARAGGPG |
| Persephin-full | -MAVGKFLLGSLLLLSLQLGQGWGPDARGVPVADGEFSSEQVAKAGGTWLGTHRPL |
| GDNF_HUMAN-full | NMPEDYPDQFDDVMDFIQATIKRLKRSPDKQMAVLPRRERNRQAAAANPENSRGKG |
| Neurturin-full | RARARLGARPCGLRELEVRVSELGLGYASDETVLFRYCAGACEA-AARVYDLGLRR |
| Neublastin | SRARAAGARGCRLRSQLVPVRALGLGHRSDELVRFRFCSGSCRR-ARSPHDLSLAS |
| Persephin-full | ARLRRALSGPCQLWSLTLSVAELGLGYASEEKVIFRYCAGSCPRGARTQHGLALAR |
| GDNF_HUMAN-full | RRGQRGKNRGCVLTAIHLNVTDLGLGYETKEELIFRYCSGSCDA-AETTYDKILKN |
| | * * : * ****: :.* : **:*:*:* * :. * |
| Neurturin-full | LRQRRRLRRE---RVRAQPCCRPTAYEDEVSFLDAHSRYHTVHELSARECACV- |
| Neublastin | LLGAGALRPPPGSRPVSQPCCRPTRYE-AVSFMDVNSTWRTVDRLSATACGCLG |
| Persephin-full | LQGQGRAHGG--------PCCRPTRYT-DVAFLDDRHRWQRLPQLSAAACGCGG |
| GDNF_HUMAN-full | LSRNRRLVSD----KVGQACCRPIAFDDDLSFLDDNLVYHILRKHSAKRCGCI- |
| | * .**** : ::*:* . :: : . ** *.* |

*indicates positions which have a single, fully conserved residue.
:indicates that one of the following ′strong′ groups is fully conserved:
-STA, NEQK, NHQK, NDEQ, QHRK, MILV, MILF, HY, FYW.
.indicates that one of the following ′weaker′ groups is fully conserved:
-CSA, ATV, SAG, STNK, STPA, SGND, SNDEQK, NDEQHK, NEQHRK, HFY.

From the amino acid sequence alignment shown in Table 1, it can be seen that neublastin has seven cysteine residues at locations that are conserved within the TGF-β superfamily. Based on this sequence alignment, neublastin was shown to be a member of the GDNF subfamily of neurotrophic factors (LGLG-FR(Y/F)CSGSC-QxCCRP-SAxxCGC, the GDNF subfamily fingerprint, underlined in Table 1).

The neublastin polypeptides useful herein may be provided in any bioactive form, including the form of pre-pro-proteins, pro-proteins, mature proteins, glycosylated proteins, phosphorylated proteins, truncated forms, or any other post-translationally modified protein. It is assumed that a bioactive neublastin is in the dimerized form for each NBN variant, because dimer formation is required for activity. Little to no activity is observed in a monomeric NBN polypeptide. A bioactive neublastin polypeptide includes a dimerized polypeptide that, in the presence of a cofactor (such as GFRα3 or RET), binds to GFRα3 or to a complex of GFRα3 and RET, induces dimerization of RET, and autophosphorylation of RET. Accordingly, a “neublastin polypeptide,” as used herein, is a polypeptide which possesses neurotrophic activity (e.g., as described in WO 00/01815) as follows:

### 1. Wild-Type Neublastin

The following “wild-type” neublastin amino acid (“aa” or “AA”) sequences are exemplary of those that are useful in the methods and compositions of this invention:

AA₋₈₀-AA₁₄₀ of SEQ ID NO:2 (“wild type” human prepro),

AA₋₄₁-AA₁₄₀ of SEQ ID NO:2 (pro human),

AA₁-AA₁₄₀ of SEQ ID NO:2 (mature 140AA (SEQ ID NO:11); hereafter “140NBN”),

AA₂₅-AA₁₄₀ of SEQ ID NO:2 (mature 116AA (SEQ ID NO:12); hereafter “116NBN”),

AA₂₈-AA₁₄₀ of SEQ ID NO:2 (mature 113AA (SEQ ID NO:13); hereafter “113NBN”),

AA₋₈₀-AA₁₄₄ of SEQ ID NO:4 (murine prepro),

AA₁-AA₁₄₄ of SEQ ID NO:4 (murine mature—144 AA),

AA₁-AA₂₂₄ of SEQ ID NO:5 (rat prepro),

AA₈₁-AA₂₂₄ of SEQ ID NO:5 (rat mature—144 AA),

Peptides with a C-terminal sequence set forth in AA₁₀₇-AA₁₄₀ of SEQ ID NO:2, more preferably AA₇₆-AA₁₄₀ of SEQ ID NO:2, and which retain the 7 Cys residues characteristic of the GDNF family and of the TGF-β super family.

In one embodiment, the preferred neublastin polypeptide contains (seven) cysteines conserved as in SEQ ID NO:2 at positions 43, 70, 74, 107, 108, 136 and 138. These seven conserved cysteine residues are known within the TGF-superfamily to form three intramonomeric disulfide bonds (contemplated, e.g., in SEQ ID NO:2 between cysteine residues 43-108, 70-136, and 74-138) and one intermonomeric disulfide bond (contemplated, e.g., in SEQ ID NO:2 between cysteine residues 107-107), which together with the extended beta strand region constitutes the conserved structural motif for the TGF-β superfamily. See, e.g., Daopin et al., Proteins 1993, 17:176-192.

### 2. Truncated Neublastins (“NBNs”)

Neublastin polypeptides useful in the present invention also include truncated forms of the full length neublastin molecule. In such truncated molecules, one or more amino acids have been deleted from the N-terminus or the C-terminus, preferably the N-terminus. The truncated neublastin polypeptide may be obtained by providing a mature neublastin polypeptide and contacting the mature neublastin polypeptide with at least one protease under conditions sufficient to produce the truncated neublastin polypeptide. Preferably, at least one protease is an exoprotease, and contacting the mature neublastin polypeptide results in formation of an exopeptidase neublastin polypeptide digestion product that can be further digested with a dipeptidyl peptidase.

The truncated neublastin polypeptides described herein preferably include a polypeptide sequence that encompasses the seven cysteine residues conserved in the mature neublastin sequence. In certain preferred embodiments, the truncated neublastin polypeptide includes at least the 85 carboxy terminal amino acids of mature 113NBN neublastin polypeptide.

Other variants of Neublastin include truncated NBN forms. Examples of these include:
(i) the 112AA polypeptide sequence designated herein as NBN112, which possesses the carboxy terminal 112 amino acids of a mature neublastin polypeptide, e.g., amino acids 29-140 of SEQ ID NO:2 (SEQ ID NO:14).
(ii) the 111AA polypeptide sequence designated herein as NBN111, which possesses the carboxy terminal 111 amino acids of a mature neublastin polypeptide, e.g., amino acids 30-140 of SEQ ID NO:2 (SEQ ID NO:15).
(iii) the 110AA polypeptide sequence designated herein as NBN110, which possesses the carboxy terminal 110 amino acids of a mature neublastin polypeptide, e.g., amino acids 31-140 of SEQ ID NO:2 (SEQ ID NO:16).
(iv) the 109AA polypeptide sequence designated herein as NBN109, which possesses the carboxy terminal 109 amino acids of a mature neublastin polypeptide, e.g., amino acids 32-140 of SEQ ID NO:2 (SEQ ID NO:17).
(v) the 108AA polypeptide sequence designated herein as NBN108, which possesses the carboxy terminal 108 amino acids of a mature neublastin polypeptide, e.g., amino acids 33-140 of SEQ ID NO:2 (SEQ ID NO:18).
(vi) the 107AA polypeptide sequence designated herein as NBN107, which possesses the carboxy terminal 107 amino acids of a mature neublastin polypeptide, e.g., amino acids 34-140 of SEQ ID NO:2 (SEQ ID NO:19).
(vii) the 106AA polypeptide sequence designated herein as NBN106, which possesses the carboxy terminal 106 amino acids of a mature neublastin polypeptide, e.g., amino acids 35-140 of SEQ ID NO:2 (SEQ ID NO:20).
(viii) the 105AA polypeptide sequence designated herein as NBN105, which possesses the carboxy terminal 105 amino acids of a mature neublastin polypeptide, e.g., amino acids 36-140 of SEQ ID NO:2 (SEQ ID NO:21).
(ix) the 104AA polypeptide sequence designated herein as NBN104, which possesses the carboxy terminal 104 amino acids of a mature neublastin polypeptide, e.g., amino acids 37-140 of SEQ ID NO:2 (SEQ ID NO:22).
(x) the 103AA polypeptide sequence designated herein as NBN103, which possesses the carboxy terminal 103 amino acids of a mature neublastin polypeptide, e.g., amino acids 38-140 of SEQ ID NO:2 (SEQ ID NO:23).
(xi) the 102AA polypeptide sequence designated herein as NBN102, which possesses the carboxy terminal 102 amino acids of a mature neublastin polypeptide, e.g., amino acids 39-140 of SEQ ID NO:2 (SEQ ID NO:24).
(xii) the 101AA polypeptide sequence designated herein as NBN101, which possesses the carboxy terminal 101 amino acids of a mature neublastin polypeptide, e.g., amino acids 40-140 of SEQ ID NO:2 (SEQ ID NO:25).
(xiii) the 100AA polypeptide sequence designated herein as NBN100, which possesses the carboxy terminal 100 amino acids of a mature neublastin polypeptide, e.g., amino acids 41-140 of SEQ ID NO:2 (SEQ ID NO:26).
(xiv) the 99AA polypeptide sequence designated herein as NBN99, which possesses the carboxy terminal 99 amino acids of a mature neublastin polypeptide, e.g., amino acids 42-140 of SEQ ID NO:2 (SEQ ID NO:27).

It is understood that the truncated forms of Neublastin disclosed herein (e.g., the 112AA through 99AA forms) have neurotrophic activity.

In most preferred embodiments, the truncated neublastin polypeptide is the 99 aa, 100 aa, 101 aa, 102 aa, 103 aa, 104 aa, 105 aa, 106 aa, 107 aa, 108 aa, 109 aa, 110 aa, 111 aa or 112 aa carboxy terminal amino acids of mature 113 AA neublastin polypeptide (i.e., NBN99, NBN100, NBN101, NBN102, NBN103, NBN104, NBN105, NBN106, NBN107, NBN108, NBN109, NBN110, NBN111 or NBN112, respectively). The sequences may also be found in the murine and rat neublastin polypeptides as the carboxy terminal 99 aa, 100 aa, 101 aa, 102 aa, 103 aa, 104 aa, 105 aa, 106 aa, 107 aa, 108 aa, 109 aa, 110 aa, 111 aa or 112 aa, respectively, in SEQ ID NOS:4 and 5. These most preferred examples of truncated NBN forms are bioactive (referred to “bioactive truncated neublastin polypeptides”) as they have been demonstrated herein to have neurotrophic activity. As stated above, NBN dimerization is required for bioactivity, as little to no activity is observed with the NBN monomeric polypeptide.

### 3. Variant Neublastins (“NBNs”) With Substantial Similarity or Identity

The NBNs useful in this invention also include those NBN polypeptides that have an amino acid sequence with substantial similarity or identity to the various prepro, pro, mature and truncated “neublastin” polypeptides set forth above. Preferably the neublastin polypeptide used has at least 70%, more preferably 85%, still more preferably 90%, or still further preferably 95% identity or similarity to the neublastin polypeptides in SEQ ID NOS:2, 4, 5 or 11-27. Most preferably the neublastin polypeptide used has at least 99% similarity or identity to the neublastin polypeptides in SEQ ID NOS:2, 4, 5 or 11-27.

The degree to which a candidate polypeptide shares homology with a neublastin polypeptide of the invention is determined as the degree of similarity or identity between two amino acid sequences.

A high level of sequence identity indicates a likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity is determined by computer analysis, such as, without limitations, the ClustalX computer alignment program (Thompson et al., Nucleic Acids Res. 1997, 25(24):4876-82), and the default parameters suggested therein. Using this program, the mature part of a polypeptide encoded by an analogous DNA sequence of the invention exhibits a degree of identity of at least 70%, more preferably 85%, still more preferably 90%, or still further preferably 95%, most preferably at least 99% with the amino acid sequences presented herein as SEQ ID NO:2 (human NBN), SEQ ID NOS:4 and 5 (rodent NBN), or SEQ ID NOS:11-27 (mature and truncated NBN).

Other alignment tools are known, such as the dynamic programming algorithm described in Needleman et al., J. Mol. Biol. 48:443 (1970), and the Align Program, a commercial software package produced by DNAstar, Inc. the teachings of which are incorporated by reference herein. Once the alignment between the candidate and reference sequence is made and refined, a percent homology score is calculated. The individual amino acids of each sequence are compared sequentially according to their similarity to each other.

Similarity factors include similar size, shape and electrical charge. One particularly preferred method of determining amino acid similarities is the PAM25O matrix described in Dayhoff et al., ATLAS OF PROTEIN SEQUENCE AND STRUCTURE 345-352 (1978 & Supp.), incorporated by reference herein. A similarity score is first calculated as the sum of the aligned pairwise amino acid similarity scores. Insertions and deletions are ignored for the purposes of percent homology and identity. Accordingly, gap penalties are not used in this calculation. The raw score is then normalized by dividing it by the geometric mean of the scores of the candidate compound and the seven cysteine skeleton of the neublastin polypeptides. The geometric mean is the square root of the product of these scores. The normalized raw score is the percent homology.

As noted above, the neublastin polypeptides of the invention include variant polypeptides. In the context of this invention, the term “variant polypeptide” includes a polypeptide (or protein) having an amino acid sequence that differs from the sequences presented as SEQ ID NO:2 (human NBN), or SEQ ID NOS:4 and 5 (rodent NBN), or SEQ ID NOS:11-27 (mature and truncated NBN), at one or more amino acid positions. Such variant polypeptides include the modified polypeptides described above, as well as conservative substitutions, splice variants, isoforms, homologues from other species, and polymorphisms.

As defined herein, the term “conservative substitutions” denotes the replacement of an amino acid residue by another, biologically similar, residue. Typically, biological similarity, as referred to above, reflects substitutions on the wild type sequence with conserved amino acids. For example, one would expect conservative amino acid substitutions to have little or no effect on the biological activity, particularly if they represent less than 10% of the total number of residues in the polypeptide or protein. Preferably, conservative amino acid substitutions represent changes in less than 5% of the polypeptide or protein, most preferably less than 2% of the polypeptide or protein. For example, when calculated in accordance, e.g., with human 113NBN, most preferred conservative substitutions would represent fewer than three amino acid substitutions in the wild type mature amino acid sequence. In a particularly preferred embodiment, there is a single amino acid substitution in the mature sequence, wherein both the substituted and replacement amino acid are non-cyclic.

Other examples of particularly conservative substitutions include the substitution of one hydrophobic residue for another, such as isoleucine, valine, leucine or methionine, or the substitution of one polar residue for another, such as the substitution of arginine for lysine, glutamic for aspartic acid, or glutamine for asparagine, and the like.

The term conservative substitution also includes the use of a substituted amino acid residue in place of an un-substituted parent amino acid residue provided that antibodies raised to the substituted polypeptide also immunoreact with the un-substituted polypeptide.

Modifications of this primary amino acid sequence may result in proteins which have substantially equivalent activity as compared to the unmodified counterpart polypeptide, and thus may be considered functional analogs of the parent proteins. Such modifications may be deliberate, e.g. as by site-directed mutagenesis, or they may occur spontaneously, and include splice variants, isoforms, homologues from other species, and polymorphisms. Such functional analogs are also contemplated according to the invention.

Moreover, modifications of the primary amino acid sequence may result in proteins which do not retain the biological activity of the parent protein, including dominant negative forms, etc. A dominant negative protein may interfere with the wild-type protein by binding to, or otherwise sequestering regulating agents, such as upstream or downstream components, that normally interact functionally with the polypeptide. Such dominant negative forms are also contemplated according to the invention.

### 4. Derivative or Modified NBNs

Polypeptides of the present invention also include chimeric polypeptides or cleavable fusion polypeptides in which another polypeptide is fused at the N-terminus or the C-terminus of the polypeptide or fragment thereof. A chimeric polypeptide may be produced by fusing a nucleic acid sequence (or a portion thereof) encoding another polypeptide to a nucleic acid sequence (or a portion thereof) of the present invention. Techniques for producing chimeric polypeptides are standard techniques. Such techniques usually require joining the sequences such that they are in the same reading frame, and expression of the fused polypeptide under the control of the same promoter(s) and terminator.

The neublastin polypeptides may be N-glycosylated polypeptides. In one embodiment, the Asn residue at position 122 of SEQ ID NO:2 is glycosylated.

This invention also contemplates neublastin fusion proteins, such as Ig-fusions, as described, e.g., in U.S. Pat. Nos. 5,434,131; 5,565,335; 5,541,087; and 5,726,044, each herein incorporated by reference, or preferably serum albumin fusions.

The neublastin polypeptides useful here include timed-release compositions and neublastin polypeptides modified with a derivative moiety (preferably a polyethylene glycol moiety) to have an extended residence time and/or increased concentration in body fluids. In addition, the neublastin polypeptide may be derivatized with a moiety selected from the group consisting of aliphatic esters, amides, N-acyl-derivatives, or O-acyl derivatives.

Table 2 below shows a ClustalW comparison between human (SEQ ID NO:2), mouse (SEQ ID NO:4) and rat (SEQ ID NO:5) prepro-neublastin. Mature NBN polypeptides NBN140, NBN116, NBN113 and NBN99 are indicated by “*”. The N-terminus of NBN112 through NBN100 are indicated by consecutive “:” symbols. Table 3 below shows distance comparison between wild type human, mouse and rat neublastin polypeptides SEQ ID NOS:2, 4 and 5).

TABLE 2
ClustalW comparison of Human, Mouse and Rat Neublastin

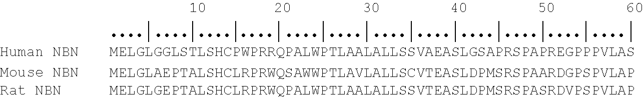

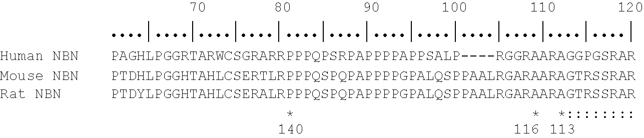

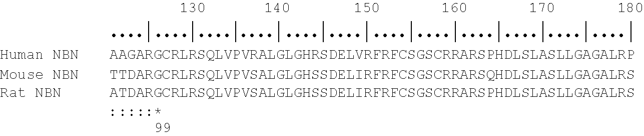

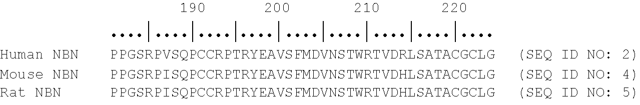

TABLE 3
Distance comparison of Human, Mouse and Rat Neublastim

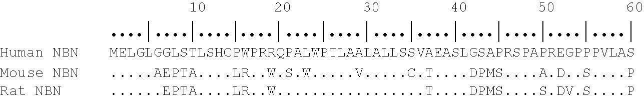

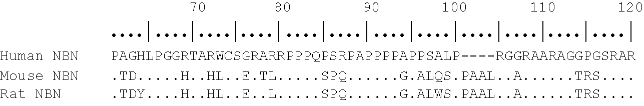

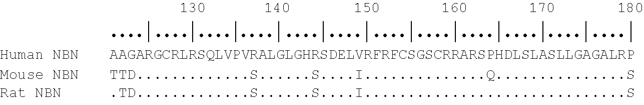

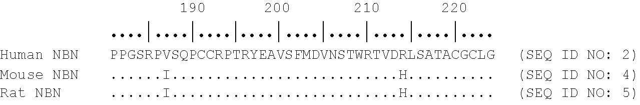

### Methods of Producing the Neublastin Polypeptide

The neublastin polypeptide used herein may be isolated from mammalian cells, preferably from a human cell or from a cell of murine origin. In a most preferred embodiment, the neublastin polypeptide may be isolated from human heart tissue, from human skeletal muscle, from human pancreas, or from human brain tissue, in particular from caudate nucleus or from thalamus, or it may be obtained from DNA of mammalian origin, as discussed in more detail below.

Alternately, the neublastin polypeptides may be obtained by expression of polynucleotides that encode such neublastin polypeptides. Such polynucleotides include DNA, cDNA and RNA sequences and are available in the art. See, e.g. WO 00/01815, WO 00/04050 and WO 00/18799, incorporated herein by reference. Particularly useful polynucleotides have the DNA sequence presented as SEQ ID NO:1 (human NBN cDNA), and the DNA sequence presented as SEQ ID NO:3 (mouse NBN cDNA). In addition, the genomic sequence for human NBN may be used (see GenBank Accession No. AC 005038).

More specifically, the neublastin polypeptides useful herein may be obtained by culturing a cell containing a nucleic acid sequence encoding a neublastin polypeptide under conditions permitting the production of the neublastin polypeptide, followed by recovery of the neublastin polypeptide from the culture medium. The nucleic acid sequence encoding a neublastin polypeptide may be a nucleic acid sequence that is normally endogenous to the cell or an exogenously-derived nucleic acid sequence that is introduced into a “production” cell. When cells are to be genetically modified for the purposes of producing a neublastin polypeptide, the cells may be modified by conventional methods or by gene activation.

According to conventional methods, a DNA molecule that contains a neublastin cDNA or genomic DNA sequence may be contained within an expression construct and transfected into cells by standard methods including, but not limited to, liposome-, polybrene-, or DEAE dextran-mediated transfection, electroporation, calcium phosphate precipitation, microinjection, or velocity driven microprojectiles (“biolistics”). Alternatively, one could use a system that delivers DNA by viral vector. Viruses known to be useful for gene transfer include adenoviruses, adeno-associated virus, lentivirus, herpes virus, mumps virus, poliovirus, retroviruses, Sindbis virus, and vaccinia virus such as canary pox virus, as well as Baculovirus infection of insect cells, in particular Sf9 insect cells.

Alternatively, the cells may be modified to produce a neublastin polypeptide using a gene activation (“GA”) approach, such as described in U.S. Pat. Nos. 5,733,761 and 5,750,376, each incorporated herein by reference.

Accordingly, the term “genetically modified,” as used herein in reference to cells, is meant to encompass cells that express a particular gene product following introduction of a nucleic acid molecule encoding the gene product and/or regulatory elements that control expression of a endogenous coding sequence for the gene product. The nucleic molecule may be introduced by gene targeting, allowing incorporation of the nucleic molecule at a particular genomic site.

In one embodiment, the neublastin polypeptide is produced in a bacterial cell, preferably E. coli. In a different embodiment, the neublastin polypeptide is produced in an insect derived cell, particularly Sf9.

In another embodiment, the neublastin polypeptide is produced in, e.g., a mammalian cell, e.g., a human cell, an oocyte, or a yeast cell. The cell of the invention may be without limitation a human embryonic kidney (“HEK”) cell, e.g., a HEK 293 cell, a BHK21 cell, a Chinese hamster ovary (“CHO”) cell, a Xenopus laevis oocyte (“XLO”) cell, or Pichia pastoris (yeast). In one embodiment, the cell of the invention is a fungal cell, e.g., a filamentous fungal cell. In yet another embodiment, the cell is an insect cell, most preferably the SD cell. Additional mammalian cells of the invention are PC12, HiB5, RN33b cell lines, human neural progenitor cells, and other cells derived from human cells, especially neural cells.

Examples of primary or secondary cells include fibroblasts, epithelial cells including mammary and intestinal epithelial cells, endothelial cells, formed elements of the blood including lymphocytes and bone marrow cells, glial cells, hepatocytes, keratinocytes, muscle cells, neural cells, or the precursors of these cell types. Examples of immortalized human cell lines useful in the present methods include, but are not limited to, Bowes Melanoma cells (ATCC Accession No. CRL 9607), Daudi cells (ATCC Accession No. CCL 213), HeLa cells and derivatives of HeLa cells (ATCC Accession Nos. CCL 2, CCL 2.1, and CCL 2.2), HL-60 cells (ATCC Accession No. CCL 240), HT-1080 cells (ATCC Accession No. CCL 121), Jurkat cells (ATCC Accession No. TIB 152), KB carcinoma cells (ATCC Accession No. CCL 17), K-562 leukemia cells (ATCC Accession No. CCL 243), MCF-7 breast cancer cells (ATCC Accession No. BTH 22), MOLT-4 cells (ATCC Accession No. 1582), Namalwa cells (ATCC Accession No. CRL 1432), Raji cells (ATCC Accession No. CCL 86), RPMI 8226 cells (ATCC Accession No. CCL 155), U-937 cells (ATCC Accession No. CRL 1593), WI-38VA13 sub line 2R4 cells (ATCC Accession No. CLL 75.1), and 2780AD ovarian carcinoma cells (Van der Blick et al., Cancer Res. 48:5927-5932, 1988), as well as heterohybridoma cells produced by fusion of human cells and cells of another species. Secondary human fibroblast strains, such as WI-38 (ATCC Accession No. CCL 75) and MRC-5 (ATCC Accession No. CCL 171), may also be used.

When the cell is an eukaryotic cell, incorporation of the heterologous polynucleotide of the invention may in particular be carried out by infection (employing a virus vector), by transfection (employing a plasmid vector), using calcium phosphate precipitation, microinjection, electroporation, lipofection, or other physical-chemical methods known in the art.

The NBN polypeptides are isolated from production cell cultures, or from culture medium conditioned by production cells, using standard protein purification techniques including refolding if applicable. Suitable techniques are described below in the Examples.

### Subjects for Treatment

This invention can be used for the treatment or prophylaxis of neuropathic pain, including tactile allodynia, and for reducing loss of pain sensitivity associated with neuropathy, in a mammalian subject afflicted therewith, or at risk thereof. Subjects at risk of developing a neuropathy and at risk of loss of pain sensitivity associated with such neuropathy include subjects with diabetes, subjects who are receiving chemotherapy, subjects who have experienced certain traumas, subjects who have ingested various toxins or drugs, subjects experiencing certain vitamin deficiencies, subjects infected with certain viral pathogens, subjects afflicted with various autoimmune disorders and metabolic disorders, and subjects who have experienced certain nerve damage or neurodegeneration. Mammalian subjects include sheep, horses, dogs, cats, pigs, rabbits, guinea pigs, rats, hamsters, gerbils and mice, but most preferably are humans.

Typically, in human subjects, the patient is either refractory to other traditional pain therapies, or the subject responds insufficiently to other such pain therapies to provide satisfactory pain control.

In general, this invention features both prophylactic treatment and therapeutic treatment protocols. In prophylactic treatment, a neublastin polypeptide is administered to a subject at risk of neuropathic pain or developing loss of pain sensitivity; such subjects would be expected to be subjects with an early stage neuropathy. The treatment with neublastin under such circumstances would serve to treat at-risk patients preventively.

In therapeutic treatment, a neublastin polypeptide is administered to a subject who has experienced neuropathic pain or who has experienced loss of pain sensitivity as a result of affliction with a neuropathy; such subjects would be expected to be subjects with a late stage neuropathy. The treatment with neublastin under such circumstances would serve to alleviate the neuropathic pain and/or rescue appropriate pain sensitivity in the subject. Such late stage patients may have received a number of therapies, beginning with self-medication (such as non-steroidal anti-inflammatory drugs, e.g., ibuprofen). Such treatments may be escalated to antidepressants (e.g., tricyclic antidepressants, vanlafaxine, and selective serotonin re-uptake inhibitors—specific medications include amitriptyline, desipramine, imipramine and nortriptyline), or anticonvulsants (e.g., gabapentin, carbamazepine, lamotrigine, topiramate, and phenyloin). Other medication include topical analgesics (e.g., capsaicin, and lidoderm), anti-arrhythmics and opioids. Surgery may be performed in severe neuropathy cases. Studies indicate that fewer than 50% of patients respond to topical analgesics, anti-arrhythmics, opioids or surgery.

### Methods and Pharmaceutical Compositions

This invention provides methods for treating neuropathic pain, for treating tactile allodynia, and for reducing loss of pain sensitivity associated with neuropathy. The present methods use neublastin polypeptides, including full-length neublastin polypeptides or bioactive truncated neublastin polypeptides. In addition, the invention provides pharmaceutical compositions containing a full length neublastin polypeptide or a truncated neublastin polypeptide suspended, dissolved, or dispersed in a pharmaceutically acceptable carrier.

### 1. Treatment of Neuropathic Pain

In one embodiment, the invention features a method for treating neuropathic pain in a subject comprising administering to the subject an effective amount of a neublastin polypeptide. The neublastin polypeptide may be administered alone (mono-therapy) or as part of a combination therapy regime. Preferred combination therapies include administering to the subject an effective amount of an analgesia-inducing compound selected from the group consisting of opioids, anti-arrhythmics, topical analgesics, local anaesthetics, anticonvulsants, antidepressants, corticosteroids and non-steroidal anti-inflammatory drugs (NSAIDS).

The neublastin polypeptides and nucleic acids of this invention (and pharmaceutical compositions containing same described herein) are used in the treatment of pain associated with peripheral neuropathies. Among the peripheral neuropathies which can be treated according to this invention are trauma-induced neuropathies, e.g., those caused by physical injury or disease state, physical damage to the brain, physical damage to the spinal cord, stroke associated with brain damage, and neurological disorders related to neurodegeneration.

The invention also provides treatments of chemotherapy-induced neuropathies (such as those caused by delivery of chemotherapeutic agents, e.g., taxol or cisplatin); toxin-induced neuropathies, drug-induced neuropathies, pathogen-induced (e.g., virus induced) neuropathies, vitamin-deficiency-induced neuropathies; idiopathic neuropathies; and diabetic neuropathies. See, e.g., U.S. Pat. Nos. 5,496,804 and 5,916,555, each herein incorporated by reference. The invention still further can be used for treatment of mono-neuropathies, mono-multiplex neuropathies, and poly-neuropathies, including axonal and demyelinating neuropathies, using the neublastin nucleotides and polypeptides of this invention.

The neuropathic pain may be associated with a number of peripheral neuropathies, including:

(a) trauma-induced neuropathies,

(b) chemotherapy-induced neuropathies,

(c) toxin-induced neuropathies (including but not limited to neuropathies induced by alcoholism, vitamin B6 intoxication, hexacarbon intoxication, amiodarone, chloramphenicol, disulfuram, isoniazide, gold, lithium, metronidazole, misonidazole, nitrofurantoin),

(d) drug-induced neuropathies, including therapeutic drug-induced neuropathic pain (such as caused by anti-cancer agents, particularly anti-cancer agents selected from the group consisting of taxol, taxotere, cisplatin, nocodazole, vincristine, vindesine and vinblastine; and such as caused by anti-viral agents, particularly anti-viral agents selected from the group consisting of ddI, DDC, d4T, foscarnet, dapsone, metronidazole, and isoniazid).

(e) vitamin-deficiency-induced neuropathies (including but not limited to vitamin B12 deficiency, vitamin B6 deficiency, and vitamin E deficiency),

(f) idiopathic neuropathies,

(g) diabetic neuropathies,

(h) pathogen-induced nerve damage,

(i) inflammation-induced nerve damage,

(j) neurodegeneration,

(k) hereditary neuropathy (including but not limited to Friedreich ataxia, familial amyloid polyneuropathy, Tangier disease, Fabry disease),

(l) metabolic disorders (including but not limited to renal insufficiency and hypothyroidism),

(m) infectious and viral neuropathies (including but not limited to neuropathic pain associated with leprosy, Lyme disease, neuropathic pain associated with infection by a virus, particularly a virus selected from the group consisting of a herpes virus (e.g. herpes zoster which may lead to post-herpetic neuralgia), a human immunodeficiency virus (HIV), and a papilloma virus),

(n) auto-immune neuropathies (including but not limited to Guillain-Barre syndrome, chronic inflammatory de-myelinating polyneuropathy, monoclonal gammopathy of undetermined significance and polyneuropathy),

(o) trigeminal neuralgia and entrapment syndromes (including but not limited to Carpel tunnel),

(p) other neuropathic pain syndromes including post-traumatic neuralgia, phantom limb pain, multiple sclerosis pain, complex regional pain syndromes (including but not limited to reflex sympathetic dystrophy, causalgia), neoplasia-associated pain, vasculitic/angiopathic neuropathy, and sciatica.

Neuropathic pain may be manifested as allodynia, hyperalgesic pain, thermal hyperalgesia, or phantom pain.

### 2. Treatment of Tactile Allodynia

The term “tactile allodynia” typically refers to the condition in a subject where pain is evoked by stimulation of the skin (e.g. touch) that is normally innocuous. This invention features a method for treating tactile allodynia in a subject.

In one embodiment, tactile allodynia is treated by administering to the subject an effective amount of a neublastin polypeptide alone.

In a second embodiment, tactile allodynia is treated by administering to the subject an effective amount of a neublastin polypeptide in combination with an effective amount of an analgesia-inducing compound selected from the group consisting of opioids, anti-arrhythmics, topical analgesics, local anaesthetics, anticonvulsants, antidepressants, corticosteroids and NSAIDS. In a preferred embodiment, the analgesia-inducing compound is an anticonvulsant. In another preferred embodiment, the analgesia-inducing compound is gabapentin ((1-aminomethyl)cyclohexane acetic acid) or pregabalin (S-(+)-4-amino-3-(2-methylpropyl)butanoic acid).

### 3. Treatment for Reduction of Loss of Pain Sensitivity

In another embodiment, the invention features a method for reducing the loss of pain sensitivity in a subject afflicted with a neuropathy. In a preferred embodiment, the neuropathy is diabetic neuropathy. In a preferred embodiment, the loss of pain sensitivity is a loss in thermal pain sensitivity. This invention contemplates both prophylactic and therapeutic treatment.

In prophylactic treatment, a neublastin polypeptide is administered to a subject at risk of developing loss of pain sensitivity; such subjects would be expected to be subjects with an early stage neuropathy. The treatment with neublastin under such circumstances would serve to treat at-risk patients preventively.

In therapeutic treatment, a neublastin polypeptide is administered to a subject who has experienced loss of pain sensitivity as a result of affliction with a neuropathy; such subjects would be expected to be subjects with a late stage neuropathy. The treatment with neublastin under such circumstances would serve to rescue appropriate pain sensitivity in the subject.

### 4. Treatment of Viral Infections and Viral-Associated Neuropathies

Prophylactic treatment of infectious and viral neuropathies is contemplated. Prophylactic treatment is indicated after determination of viral infection and before onset of neuropathic pain. During treatment, NBN polypeptide is administered to prevent appearance of neuropathic pain including but not limited to neuropathic pain associated with leprosy, Lyme disease, neuropathic pain associated with infection by a virus, particularly a virus selected from the group consisting of a herpes virus (and more particularly by a herpes zoster virus, which may lead to post-herpetic neuralgia), a human immunodeficiency virus (HIV), and a papilloma virus). In an alternative embodiment, NBN polypeptide is administered to reduce the severity of neuropathic pain, should it appear.

Symptoms of acute viral infection often include the appearance of a rash. Other symptoms include, for example, the development of persistent pain in the affected area of the body, which is a common complication of a herpes zoster infection (shingles). Post-herpetic neuralgia can last for a month or more, and may appear several months after any rash-like symptoms have disappeared. Post-herpetic neuralgia may be very severe and prolonged, and can be very resistant to treatment.

### 5. Treatment of Diabetic Neuropathies

Prophylactic treatment of diabetes associated neuropathies is contemplated. Prophylactic treatment of diabetic neuropathies would commence after determination of the initial diagnosis of diabetes or diabetes-associated symptoms and before onset of neuropathic pain. Prophylactic treatment of diabetic neuropathies may also commence upon determining that a subject is at risk for developing diabetes or diabetes-associated symptoms. During treatment, NBN polypeptide is administered to prevent appearance of neuropathic pain or reduce the severity of neuropathic pain, should it appear.

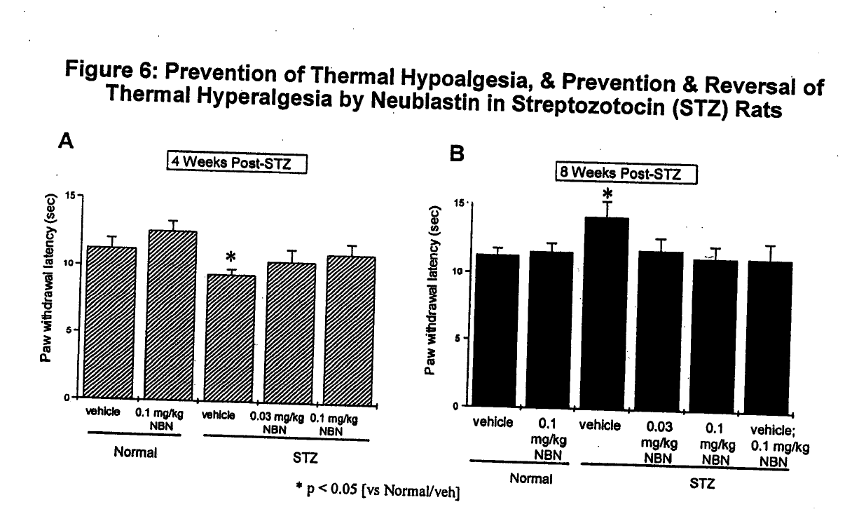

Results in FIG. 6A and FIG. 6B are relevant to treatment of diabetic neuropathy in human patients in need of such treatment. Prevention of thermal hypoalgesia and prevention and reversal of thermal hyperalgesia are contemplated, as described in Example 6.

### 6. Dosage

The foregoing methods contemplate administering to the subject, preferably systemically, a formulation comprising a neublastin polypeptide at a dosage of between 1 μg/kg to 30,000 μg/kg body weight of the subject, per dose; preferably the dosage is between 10 μg/kg to 10,000 μg/kg body weight of the subject, per dose; most preferably the dosage is between 25 μg/kg to 3,000 μg/kg body weight of the subject, per dose.

Various dosing regimes for treatment or prevention of tactile allodynia are contemplated. In one embodiment, methods of administering to a subject a formulation comprising a neublastin polypeptide include administering NBN at a dosage of between 1 μg/kg to 30,000 μg/kg body weight of the subject, per dose. In another embodiment, the dosage is between 10 μg/kg to 30,000 μg/kg body weight of the subject, per dose. In a further embodiment, the dosage is between 10 μg/kg to 10,000 μg/kg body weight of the subject, per dose. In a different embodiment, the dosage is between 25 μg/kg to 10,000 μg/kg body weight of the subject, per dose. In yet another embodiment, the dosage is between 25 μg/kg to 3,000 μg/kg body weight of the subject, per dose. In a most preferable embodiment, the dosage is between 50 μg/kg to 3,000 μg/kg body weight of the subject, per dose.

Likewise, various dosing schemes for a treatment for modulating loss of pain sensitivity are contemplated. Methods of administering to a subject a formulation comprising a neublastin polypeptide include administering NBN at a dosage of between 1 μg/kg to 30,000 μg/kg body weight of the subject, per dose; preferably the dosage is between 10 μg/kg to 10,000 μg/kg body weight of the subject, per dose; most preferably the dosage is between 25 μg/kg to 3,000 μg/kg body weight of the subject, per dose.

### 7. Delivery

The neublastin polypeptide used in the foregoing methods can be administered via any suitable delivery system, and preferably from the group consisting of intravenous delivery, intramuscular delivery, intrapulmonary delivery, subcutaneous delivery, and intraperitoneal delivery, most preferably via intramuscular delivery, intravenous delivery, or subcutaneous delivery. The neublastin polypeptide used in the foregoing methods can also be administered via intrathecal delivery.

### 8. Formulation

This invention also provides novel pharmaceutical compositions comprising a therapeutically effective amount of neublastin polypeptides suspended, dissolved, or dispersed in a pharmaceutically accepted carrier.

For use in therapy the polypeptide of the invention may be administered in any convenient form. In a preferred embodiment, the polypeptide of the invention is incorporated into a pharmaceutical composition together with one or more adjuvants, excipients, carriers and/or diluents, and the pharmaceutical composition prepared by the skilled person using conventional methods known in the art. Further details on techniques for formulation and administration may be found in the latest edition of REMINGTON'S PHARMACEUTICAL SCIENCES (Maack Publishing Co., Easton, Pa.). Acceptable diluents, carriers and excipients typically do not adversely affect the recipient's homeostasis, particularly electrolyte balance. Acceptable carriers can include biocompatible, inert or bioabsorbable salts, buffering agents, oligo- or polysaccharides, polymers, viscosity-improving agents, preservatives and the like. One exemplary carrier comprises normal physiologic saline (0.15 M NaCl, pH 7.0 to 7.4). Another exemplary carrier comprises 50 mM sodium phosphate, 100 mM sodium chloride.

### 9. Regimes

The frequency of dosing for the neublastin polypeptides of this invention is within the skills and clinical judgement of physicians. Typically, the administration regime is established by clinical trials which may establish optimal administration parameters. However, the practitioner may vary such administration regimes according to the subject's age, health, weight, sex and medical status. The frequency of dosing may also vary between acute and chronic treatments for neuropathy. In addition, the frequency of dosing may be varied depending on whether the treatment is prophylactic or therapeutic.

### EXAMPLES

### Example 1

### Expression of Neublastin Polypeptide

A. Expression in E. coli

For expression and purification in bacteria, a plasmid encoding rat neublastin was expressed in E. coli as a His-tagged fusion protein with an enterokinase cleavage site immediately adjacent to the start of the mature 113 amino acid NBN sequence. The E. coli cells were grown in a 500 L fermentor and frozen cell paste was provided. The E. coli cells were lysed in a Manton Gaulin Press and the rat NBN was recovered from the insoluble washed pellet fraction.

The NBN was extracted from the pellet with guanidine hydrochloride, refolded, and the His-tag removed with enterokinase. For further purification, the product was then subjected to chromatography on Ni NTA agarose (Qiagen), and to chromatography on Bakerbond WP CBX cation exchange resin.

The resulting product was subjected to extensive characterization including analysis by SDS-PAGE, size exclusion chromatography (SEC), reverse phase HPLC, matrix assisted laser desorption/ionization mass spectrometry (MALD/IMS), peptide mapping, assessment of activity in the KIRA ELISA, and determination of endotoxin content. The purity of the NBN product as measured by SDS-PAGE and SEC was greater than 95%. The NBN product migrated under non-reducing conditions as a dimer, consistent with its predicted structure. The endotoxin content of the material is routinely less than 1 EU/mg. The specific activity of the NBN in the KIRA ELISA is approximately 10 nM. The product was formulated at 1 mg/mL in PBS pH 6.5. The material can be supplied as a frozen liquid, which is stored at −70° C.

Similar expression systems have also been constructed for human NBN. From these constructs, human NBN has been expressed in E. coli.

### B. Expression in Mammalian Cells

Construction of plasmid pJC070.14 In order to express the neublastin cDNA in Chinese hamster ovary cells, a cDNA fragment encoding the prepro form of human neublastin was inserted into the mammalian expression vector pEAG347 to generate plasmid pJC070.14. pEAG347 contains tandem SV40 early and adenovirus major late promoters (derived from plasmid pAD2beta; Norton and Coffin, Mol. Cell. Biol. 5:281, 1985), a unique NotI cloning site, followed by SV40 late transcription termination and polyA signals (derived from plasmid pCMVbeta; MacGregor and Caskey, Nucl. Acids. Res. 17:2365, 1989). In addition, pEAG347 contains a pUC19-derived plasmid backbone and a pSV2dhfr-derived dhfr for MTX selection and amplification in transfected CHO cells.

Plasmid pJC070.14 was generated in two steps. First, a fragment encoding the prepro form of human neublastin was isolated from plasmid pUbi1Z-NBN using the polymerase chain reaction with oligonucleotides KD2-824 5′AAGGAAAAAA GCGGCCGCCA TGGAACTTGG ACTTGGAGG3′ (SEQ ID NO:9), KD2-825 5′TTITTTCCTT GGCGGCCGCT CAGCCCAGGC AGCCGCAGG3′ (SEQ ID NO:10) and PFU polymerase. The fragment was cloned into the Srf-1 site of pPCR-Script Amp SK(+) to generate the plasmid pJC069. In the second step, a partial Not-1 digest was performed on plasmid pJC069 to generate a 685 bp fragment (containing the neublastin gene) which was cloned into the Not-1 site of plasmid pEAG347 to generate plasmid pJC070.14. Transcription of the neublastin gene in plasmid pJC070.14 is controlled by the adenovirus major late promoter.

Generation of CHO cell lines expressing Neublastin. 200 μg of pJC070.14 was linearized by digestion with the restriction endonuclease Mlu-1. The DNA was extracted with phenol: chloroformisoamyl alcohol (25:24:1) and ethanol precipitated. The linearized DNA was resuspended in 20 mM Hepes pH7.05, 137 mM NaCl, 5 mM KCl, 0.7 mM Na₂HPO₄, 6 mM dextrose (REBS) and introduced into ˜4E7 CHO dukx B1(dhfr−) cells (p23) by electroporation (280V and 960 μF). Following electroporation, the cells were returned to culture in α+ Modified Eagle's Medium (MEM) supplemented with 10% fetal bovine serum (FBS) for two days. The cells were then trypsinized and replated in 100 mm dishes (100,000 cells/plate) in a-MEM (lacking ribo- and deoxyribonucleosides), supplemented with 10% dialyzed FBS, for five days. The cells were subsequently split at a density of 100,000 cells/100 mm plate, and selected in 200 nM methotrexate. Resistant colonies were picked and scaled up to 6 well plates; conditioned media from each clone was screened using a specific assay for neublastin described below. The twelve clones expressing the highest level of neublastin were scaled up to T162 flasks and subsequently reassayed. These CHO cell lines produced neublastin in the range of 25 to 50 ng/ml/day.

Ternary complex assay for neublastin. The presence of neublastin was assessed in the media of CHO cell line supernatants using a modified form of a ternary complex assay described by Sanicola et al. (Proc Natl Acad Sci USA 94:6238, 1997).

Similar mammalian expression constructs have been made and similar studies have been performed for both human NBN and rat NBN. In vivo testing of NBN activity in rats have been performed. Rat studies almost exclusively were performed with rat NBN.

### Example 2

### Efficacy of Full Length Neublastin in a Nerve Ligation Animal Model of Neuropathic Pain—Prevention of Neuropathic Pain

The preventive effect of neublastin on tactile allodynia and thermal hyperalgesia was studied in the Chung L5/L6 spinal nerve ligation (“SNL”) model (Kim and Chung (1992), Pain 50:355-363. Sprague-Dawley male rats (250-300 g) were divided into four groups. One group of rats (n=7) received a sham operation, and were administered vehicle by subcutaneous injection. A second group of rats (n=7) received a sham operation and were administered rat Neublastin (1 mg/kg) by subcutaneous injection. A third group of rats (n=7) received the spinal nerve ligation, and were administered vehicle by subcutaneous injection. A fourth group of rats (n=7) received the spinal nerve ligation, and were administered rat neublastin (1 mg/kg) by subcutaneous injection. The vehicle consisted of 5 mM phosphate and 150 mM sodium chloride at pH 6.5. Neublastin or vehicle was injected 30 minutes before the spinal nerve ligation or sham operation, and then on days 2, 4, 7, 9, 11 and 14 following the ligation or operation (post-SNL). The Von Frey (Chaplan et al. (1994), J. Neurosci. Meth. 53:55-63) and Hargreave's (Hargreaves et al. (1988), Pain 32:77-88) behavioral tests were used to monitor tactile and thermal responses, respectively. These pain responses were monitored prior to the spinal nerve ligation or sham operation to establish baseline responses, and then daily for two weeks following the operation.

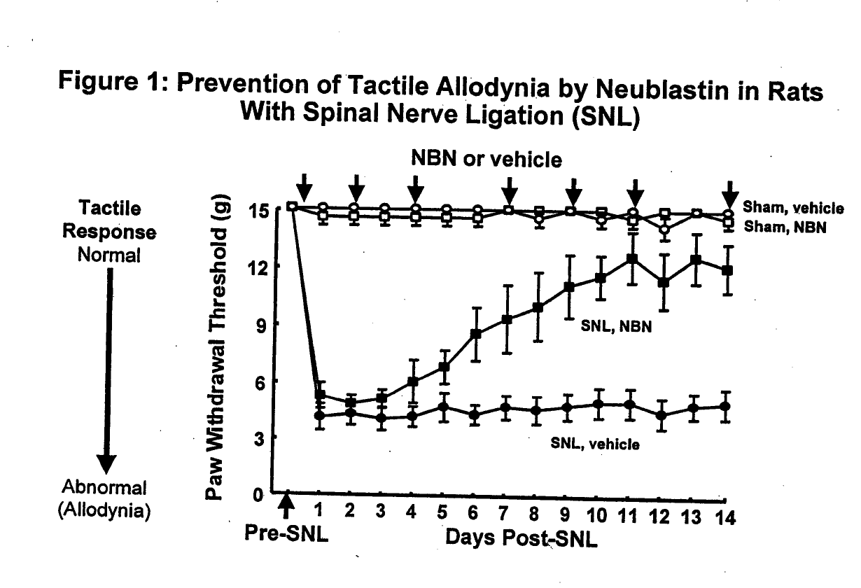

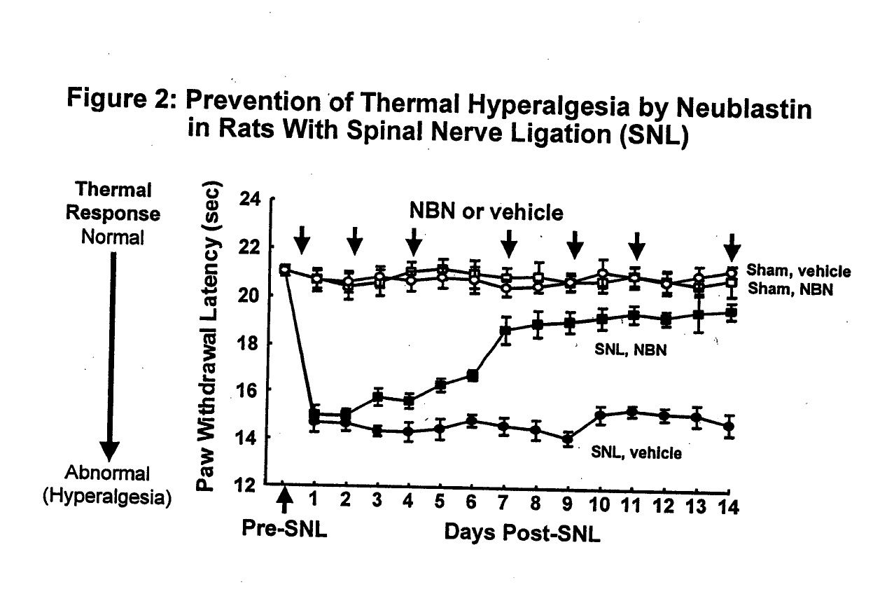

The results are depicted in FIGS. 1 and 2 as averages ±standard errors of the mean. Subcutaneous administration of neublastin (as denoted by downward arrows in FIGS. 1 and 2) led to nearly complete normalization of both types of neuropathic pain (tactile, FIG. 1 and thermal, FIG. 2) in rats with spinal nerve ligation. In sham operated rats, subcutaneous administration of neublastin did not significantly alter tactile (FIG. 1) or thermal sensitivity (FIG. 2). In spinal nerve ligated rats, the effect of neublastin on thermal sensitivity first became evident 3 days after the initiation of neublastin administration, whereas the effect on tactile allodynia first became evident slightly later, at 4-5 days after the initiation of neublastin administration. The effect of neublastin on thermal sensitivity reached a plateau approximately 7-8 days after the initiation of neublastin administration whereas the effect on tactile allodynia reached a plateau on approximately 10-11 days after the initiation of neublastin administration. The effects of neublastin did not diminish during the 2-3 day interval between administrations. In fact, there was substantial normalization of both pain behaviors between the administrations of neublastin on days 4 and 7, as measured on days 5 and 6. Following the administration of neublastin on day 11, the extant maximal normalization of both pain behaviors remained constant, suggesting that the normalization effect of neublastin on neuropathic pain is maintained for at least 3 days.

### Example 3

### Treatment of Neuropathy with a Truncated Neublastin Polypeptide

The preventive effect of a polypeptide containing the carboxy terminal 102 amino acids of mature rat neublastin in treating tactile allodynia and thermal hyperalgesia, two peripheral neuropathic conditions, is demonstrated in a Chung L5/L6 spinal nerve ligation (“SNL”) model.

Sprague-Daley male rats (approximately 250-300 grains) are divided into four groups. One group of rats (n=6) receive a sham operation and are administered vehicle by subcutaneous injection. A second group of rats (n=6) receive a sham operation and are administered truncated neublastin (1 mg/kg) by subcutaneous injection. A third group of rats (n=6) receive a spinal nerve ligation and are administered vehicle by subcutaneous ligation. A fourth group (n=6) receives the spinal nerve ligation, and are administered truncated neublastin (1 mg/kg) by subcutaneous injection. The vehicle includes 5 mM phosphate and 150 mM sodium chloride at pH 6.5. Truncated neublastin or vehicle is administered on days 0, 2, 4, 7, 9, 11 and 14. On day 0, truncated neublastin is administered 30 minutes before the spinal nerve ligation or sham operation. Pain responses are determined using behavioral tests as described in Chaplan et al. (1994), J. Neurosci. Meth. 53:55-63 and Hargreaves et al. (1988), Pain 32:77-88. These tests monitor tactile and thermal responses, respectively. The pain responses are monitored prior to the spinal nerve ligation or sham operation to establish baseline responses. Pain responses are then monitored daily for two weeks following the operation.

Subcutaneous administration of truncated neublastin is expected to lead to normalization of both types of neuropathic pain in rats with spinal nerve ligation. In sham operated rats, subcutaneous administration of truncated neublastin is not expected to significantly alter tactile or thermal sensitivity. The effect of neublastin on thermal sensitivity is predicted to become evident about 3 days after the administration of neublastin, whereas the effect on tactile allodynia is expected to first become evident slightly later.

### Example 4

### Neublastin Efficacy in a Nerve Ligation Animal Model of Neuropathic Pain—Reversal of Neuropathic Pain

The reversal effect of neublastin on tactile allodynia and thermal hyperalgesia was studied in the Chung L5/L6 spinal nerve ligation (“SNL”) model. Sprague-Dawley male rats (270-275 g) were divided into four groups. One group of rats (n=8) received a sham operation, and were administered vehicle by subcutaneous injection. A second group of rats (n=8) received a sham operation and were administered rat neublastin (1 mg/kg) by subcutaneous injection. A third group of rats (n=8) received the spinal nerve ligation, and were administered vehicle by subcutaneous injection. A fourth group of rats (n=8) received the spinal nerve ligation, and were administered rat neublastin (1 mg/kg) by subcutaneous injection. The vehicle consisted of 5 mM phosphate and 150 mM sodium chloride at pH 6.5. The Von Frey (Chaplan et al. (1994), J. Neurosci. Meth. 53: 55-63) and Hargreave's (Hargreaves et al. (1988), Pain 32: 77-88) behavioral tests were used to monitor tactile and thermal responses, respectively. These pain responses were monitored prior to the spinal nerve ligation or sham operation to establish baseline responses, and then daily for 2 weeks following the operation. Neublastin or vehicle was injected 60 minutes before behavioral testing on days 3, 5, 7, 10, 12 and 14 following the ligation or operation (post-SNL).

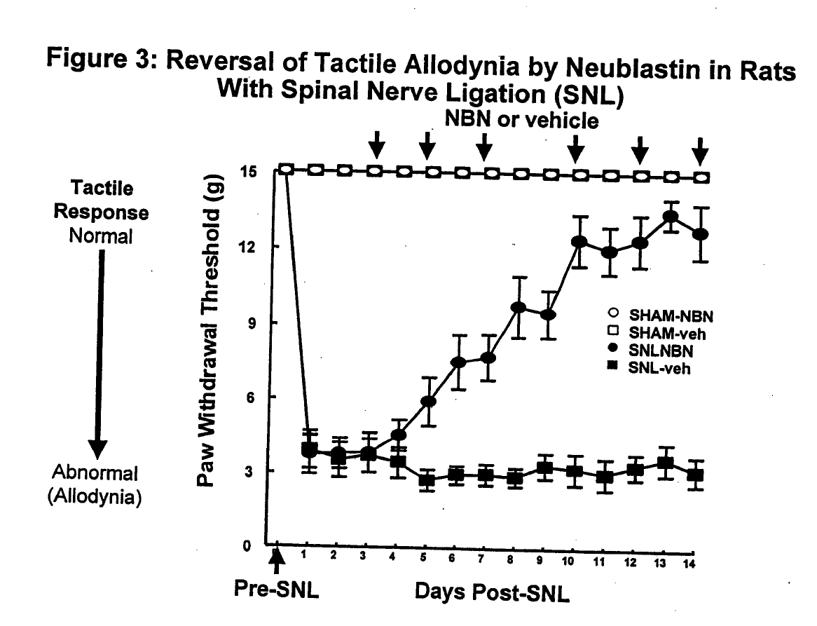

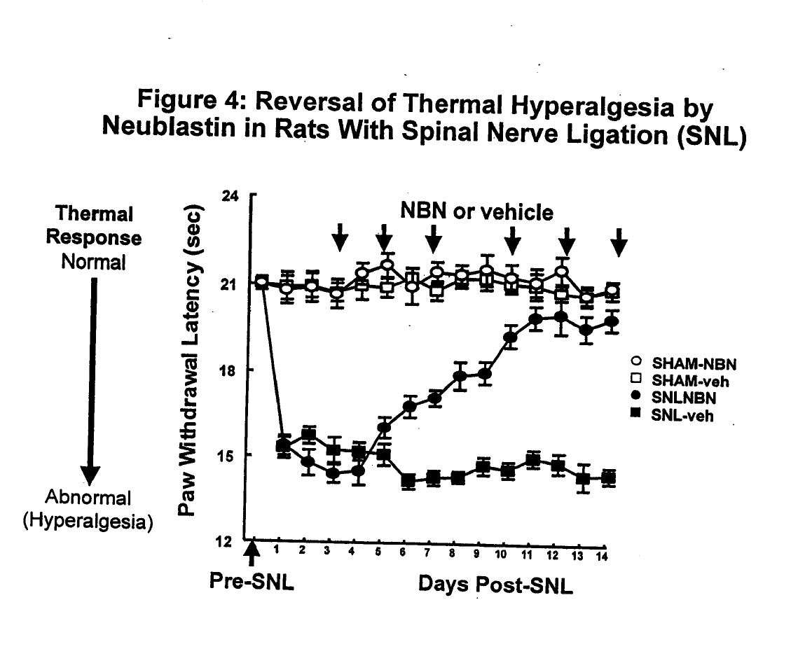

The results are depicted in FIGS. 3 and 4 as averages ±standard errors of the mean. Both types of neuropathic pain behavior (tactile allodynia shown in FIG. 3, and thermal hyperalgesia shown in FIG. 4) developed fully by day 3, as expected. Subcutaneous administration of neublastin (as denoted by downward arrows in FIGS. 3 and 4) led to nearly complete reversal of both types of neuropathic pain (tactile in FIG. 3 and thermal in FIG. 4) in rats with spinal nerve ligation, so that tactile and thermal responses were normalized. In sham operated rats, subcutaneous administration of neublastin did not significantly alter tactile (FIG. 3) or thermal (FIG. 4) sensitivity. In rats with spinal nerve ligation, the effect of neublastin on thermal sensitivity and tactile allodynia first became evident 2 to 3 days after the initiation of neublastin administration. The effect of neublastin on thermal sensitivity and tactile allodynia reached a plateau approximately 7-8 days after the initiation of neublastin administration. The effects of neublastin did not diminish during the 2 to 3 day interval between administrations. In fact, there was substantial normalization of both pain behaviors between the administrations of neublastin on days 3, 5, 7 and 10.

### Example 5

### Neublastin Efficacy in a Streptozotocin Animal Model of Neuropathic Pain—Prevention of Loss of Pain Sensitivity

The preventive effect of neublastin on loss of thermal sensitivity (thermal hypoalgesia) was studied in the streptozotocin (“STZ”) model of diabetic neuropathy. Sprague-Dawley female rats (250-275 g) were divided into 4 groups, each comprised of 10 animals. One group of rats did not receive STZ; these rats received vehicle by subcutaneous injection. Three groups of rats received 1 injection of STZ (50 mg/kg in sterile saline) and were subsequently confirmed to be hyperglycemic by spectophotometric assay of blood samples. Of the 3 groups of hyperglycemic rats, 1 group received vehicle by subcutaneous injection, a second group was administered rat neublastin (0.1 mg/kg) by subcutaneous injection, and a third group of rats was administered rat neublastin (1 mg/kg) by subcutaneous injection. The vehicle consisted of 5 mM phosphate and 150 mM sodium chloride at pH 6.5. Neublastin or vehicle was administered 3 times per week (Monday, Wednesday, Friday schedule) for 8 weeks, and was initiated at the time of STZ induction of hyperglycemia. Thermal response latencies were assessed after 8 weeks as described in Calcutt et al. (2000), Anesthesiology 93:1271-1278. In brief, a radiant heat source was applied to the plantar surface of the paw so that the surface temperature increased from 30° C. to 38.5° C. in 20 seconds, and the time latency between initiation of heating and paw withdrawal was measured to assess thermal response latency. An increase in paw withdrawal latency indicates a loss of thermal sensitivity (thermal hypoalgesia).

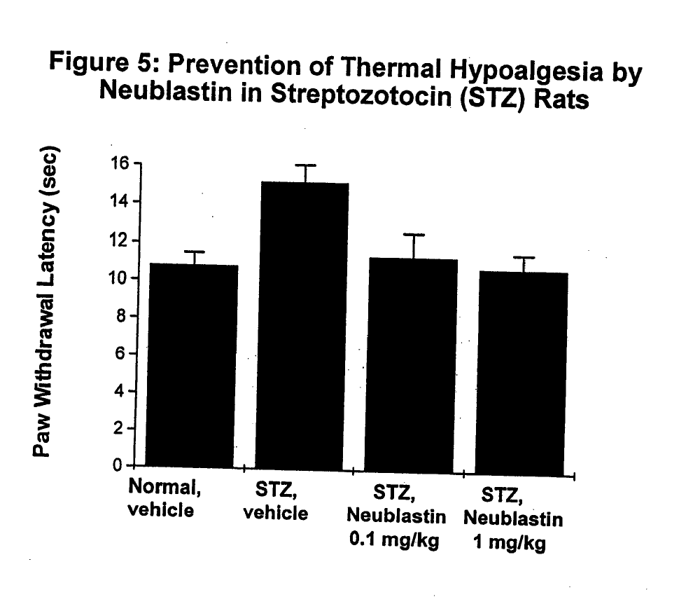

The results are depicted in FIG. 5 as averages ±standard errors of the mean. As expected at 8 weeks post-STZ, the paw withdrawal latency increased in vehicle treated STZ rats (STZ vehicle) compared to normal rats, indicating that thermal hypoalgesia had been induced by the STZ injection. Administration of neublastin prevented the increase in thermal response latency in hyperglycemic (STZ) rats. Both doses of neublastin (1 mg/kg and 0.1 mg/kg) nearly completely normalized thermal sensitivity after 8 weeks of administration at 3 times per week. These results demonstrate that neublastin prevents thermal hypoalgesia, the loss of thermal sensitivity that occurs in the STZ rat model of diabetic neuropathy.

### Example 6

### Prevention of Thermal Hypoalgesia, and Prevention and Reversal of Thermal Hyperalgesia by Neublastin in Streptozotocin Rats

Rat NBN113 was prepared in E. coli as described in Example 1. The preventive effect of neublastin on loss of thermal sensitivity (thermal hypoalgesia), and prevention and reversal by neublastin of increased thermal sensitivity (thermal hyperalgesia) was studied in the streptozotocin (“STZ”) rat model of diabetic neuropathy, as described in Example 5. Dosage studies described in Example 5 were confirmed and extended upon. Results are shown in FIG. 6A and FIG. 6B.

The results in FIG. 6A and FIG. 6B are depicted as averages ±standard errors of the mean. As expected at 4 weeks post-STZ, the paw withdrawal latency decreased in vehicle-treated STZ rats, as compared to normal rats, indicating that thermal hyperalgesia had been induced by the STZ injection. All doses of subcutaneous neublastin (0.03 mg/kg and 0.1 mg/kg) prevented thermal hyperalgesia in STZ rats after 4 weeks of administration at 3 times per week, as shown in FIG. 6A. All doses of subcutaneous neublastin (0.03 mg/kg and 0.1 mg/kg) prevented thermal hypoalgesia in STZ rats after 8 weeks of administration at 3 times per week, as shown in FIG. 6B. In addition, as shown in FIG. 6B, subcutaneous neublastin (0.1 mg/kg) administered during the second 4 wks of the 8 week treatment study (veh; 0.1 mg/kg NBN) not only reversed the thermal hyperalgesia that was apparent at 4 weeks post STZ treatment, but also prevented thermal hypoalgesia at 8 weeks from developing in STZ rats. These results demonstrate that neublastin prevents and reverses thermal hyperalgesia in the STZ rat model of diabetic neuropathy, and that neublastin prevents the loss of thermal sensitivity (thermal hypoalgesia) that occurs in the STZ rat model of diabetic neuropathy.

### Example 7

### Neublastin Efficacy in a Nerve Ligation Animal Model of Neuropathic Pain—Reversal of Neuropathic Pain is Dose Dependent

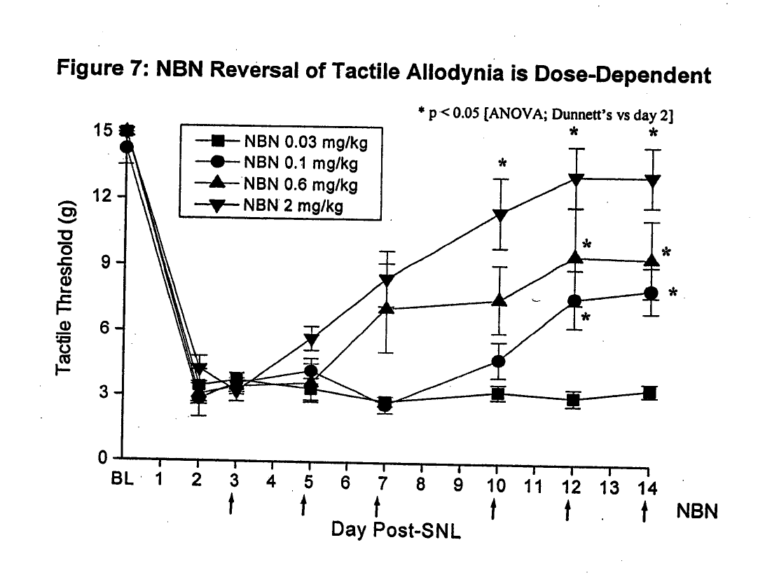

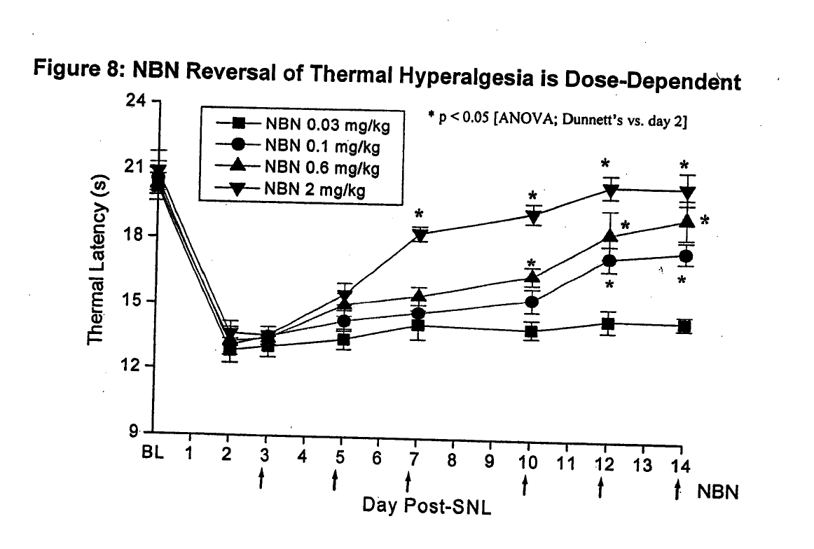

Rat NBN113 was prepared in E. coli as described in Example 1. Analysis of dose-dependent reversal by NBN113 of tactile allodynia and thermal hyperalgesia was performed in rats using the Chung L5/L6 spinal nerve ligation (“SNL”) animal model, as described in Example 4, with NBN doses of 0.03 mg/kg, 0.1 mg/kg, 0.6 mg/kg and 2 mg/kg. Experimental results shown in FIGS. 3 and 4 were confirmed and expanded upon. Results are shown in FIG. 7 and FIG. 8.

The results in FIGS. 7 and 8 are depicted as averages ±standard errors of the mean. BL indicates baseline responses. Both types of neuropathic pain behavior (tactile allodynia shown in FIG. 7, and thermal hyperalgesia shown in FIG. 8) developed fully by day 2, as expected. Subcutaneous administration of 2 mg/kg neublastin (NBN) 3 times per week (as denoted by the arrows) led to nearly complete reversal of both types of neuropathic pain (tactile in FIG. 7 and thermal in FIG. 8) in rats with spinal nerve ligation, so that tactile and thermal responses were normalized. As shown in FIG. 7, neublastin reversal of spinal nerve ligation-induced tactile allodynia was dose-dependent. Neublastin (NBN) dosing at 0.1 mg/kg s.c. or 0.6 mg/kg s.c. partially reversed tactile allodynia after 9 (as well as 11) days of dosing three times per week, whereas NBN at 2 mg/kg s.c. significantly reversed tactile allodynia after 7 (as well as 9 and 11) days of dosing three times per week, with nearly complete reversal of tactile allodynia after 9 and 11 days of dosing three times per week. Moreover, the mean reversal of spinal nerve ligation-induced tactile allodynia on day 14 post-SNL was greater with increasing subcutaneous doses of neublastin from 0.1 mg/kg to 0.6 mg/kg to 2 mg/kg. Neublastin (NBN) at 0.03 mg/kg s.c. did not significantly reverse spinal nerve ligation-induced tactile allodynia during 11 days of dosing at three times per week.

As depicted in FIG. 8, Neublastin (NBN) reversal of spinal nerve ligation (SNL)-induced thermal hyperalgesia was also dose-dependent. NBN dosing at 0.1 mg/kg significantly reversed thermal hyperalgesia after 9 (as well as 11) days of dosing three times per week, NBN at 0.6 mg/kg significantly reversed thermal hyperalgesia after 7 (as well as 9 and 11) days of dosing three times per week, and NBN at 2 mg/kg significantly reversed thermal hyperalgesia after 4 (as well as 7, 9 and 11) days of dosing three times per week. Moreover, the mean reversal of spinal nerve ligation-induced thermal hyperalgesia on day 14 post-SNL was greater with increasing subcutaneous doses of neublastin from 0.1 mg/kg to 0.6 mg/kg to 2 mg/kg. NBN at 0.03 mg/kg s.c. did not significantly reverse spinal nerve ligation-induced thermal hyperalgesia during 11 days of dosing at three times per week.

### EQUIVALENTS

Although particular embodiments have been disclosed herein in detail, this has been done by way of example for purposes of illustration only, and is not intended to be limiting with respect to the scope of the appended claims which follow. In particular, various substitutions, alterations, and modifications may be made to the invention without departing from the spirit and scope of the invention as defined by the claims.

1. A method for treating neuropathic pain in a subject, the method comprising administering to a subject in need thereof a pharmaceutical composition comprising a therapeutically effective amount of a neublastin polypeptide, wherein the pharmaceutical composition is administered to the subject via systemic delivery.

2. The method of claim 1, wherein the neuropathic pain is associated with post-herpetic neuralgia, diabetic neuropathy, or sciatica.

3. (canceled)

4. The method of claim 1, wherein the pharmaceutical composition is administered via intravenous delivery.

5. The method of claim 1, wherein the pharmaceutical composition is administered via subcutaneous delivery.

6-9. (canceled)

10. The method of claim 1, wherein the neublastin polypeptide is modified with a derivative moiety to have an extended residence time or increased concentration in body fluids.

11. The method of claim 10, wherein the derivative moiety is a polyethylene glycol moiety.

12. The method of claim 10, wherein the derivative moiety is selected from the group consisting of aliphatic esters, amides, N-acyl-derivatives, or O-acyl derivatives.

13-26. (canceled)

27. The method of claim 1, wherein said neuropathic pain is associated with infection of said subject by a virus.

28. The method of claim 27, wherein said virus is selected from the group consisting of a herpes virus, a human immunodeficiency virus (HIV), and a papilloma virus.

29. The method of claim 1, wherein said neuropathic pain is neuropathic pain associated with administration of a therapeutic agent.

30. The method of claim 29, wherein said therapeutic agent is an anti-cancer agent.

31. The method of claim 30, wherein the anti-cancer agent is selected from the group consisting of taxol, taxotere, cisplatin, nocodazole, vincristine, vindesine and vinblastine.

32. The method of claim 29, wherein said therapeutic agent is an anti-viral agent.

33. The method of claim 32, wherein said anti-viral agent is selected from the group consisting of ddI, DDC, d4T, foscarnet, dapsone, metronidazole, and isoniazid.

34. The method of claim 1, wherein said neuropathic pain is due to injury associated with trauma.

35. The method of claim 1, wherein said neuropathic pain is characterized by allodynia.

36. The method of claim 1, wherein said neuropathic pain is hyperalgesic pain.

37. The method of claim 36, wherein the hyperalgesic pain is thermal hyperalgesic pain.

38. The method of claim 1, wherein said neuropathic pain is phantom pain.

39-56. (canceled)

57. A method for treating allodynia in a subject, the method comprising administering to a subject in need thereof a pharmaceutical composition comprising a therapeutically effective amount of a neublastin polypeptide.

58. A method for treating hyperalgesic pain in a subject, the method comprising administering to a subject in need thereof a pharmaceutical composition comprising a therapeutically effective amount of a neublastin polypeptide.
